(19) United States
(12) Patent Application Publication
(10) Pub. No.: US 2026/0241070 A1
(43) Pub. Date: Aug. 20, 2026

(54) MULTIFILAMENT COLLAGEN FIBER BUNDLES WITH TENDON-LIKE STRUCTURE AND PROCESS FOR PREPARATION THEREOF

(71) Applicant: 3DBIOFIBR INC., Halifax (CA)

(72) Inventors: Hessameddin YAGHOOBIKOOPAEE, Halifax (CA); John Paul FRAMPTON, IV, Halifax (CA); Laurent KREPLAK, Halifax (CA)

(21) Appl. No.: 19/160,965

(22) PCT Filed: Feb. 28, 2024
(86) PCT No.: PCT/CA2024/050246

§ 371 Date: Aug. 29, 2025

Related U.S. Application Data
(60) Provisional application No. 63/449,344, filed on Mar. 2, 2023.

(51) Int. Cl. A61L 27/24; C07K 14/78

(52) U.S. Cl. CPC A61L 27/24; C07K 14/78; A61L 2430/10

(57) ABSTRACT

A process for producing a multifilament collagen fiber bundle involves treating a multifilament support polymer-collagen fiber bundle having a plurality of monofilaments of collagen supported on a support polymer with a series of liquid media having a decreasing concentration of the support polymer to remove the support polymer from the multifilament support polymer-collagen fiber bundle. The support polymer is more soluble than the collagen in the liquid media and the gradual removal of the support polymer provides enough time for collagen molecules to self-assemble into fibrils. The resulting multifilament collagen fiber bundle has a D-band structure similar to natural collagen but shifted by ⅙th of the D-band. The multifilament collagen fiber bundle can be crosslinked with ultraviolet light in the absence of photo-initiator to provide a crosslinked collagen product with superior tensile strength and Young's modulus making the product useful in collagen-based biomaterials for tissue repair and regeneration.

### CROSS-REFERENCE TO RELATED APPLICATIONS

This application claims the benefit of United States provisional patent application U.S. Ser. No. 63/449,344 filed Mar. 2, 2023, the entire contents of which is herein incorporated by reference.

### FIELD

This application relates to multifilament collagen fiber bundles and processes for the preparation of multifilament collagen fiber bundles.

### BACKGROUND

Tendons are fibrous tissues composed primarily of collagen. Tendon tears and ruptures are common, and can result from trauma, degenerative disease, and overuse (e.g., from physical occupations and exercise). Surgical treatment is the standard therapy for most patients suffering from debilitating tendon injuries and may entail using an artificial tendon scaffold or grafting a tendon piece obtained from another part of the body. While biodegradable synthetic biopolymer scaffolds have mechanical properties and degradation rates that make them attractive for tendon repair, natural biopolymers are more desirable because of their inherent biological activity and ability to recapitulate the biochemical and structural cues of the extracellular matrix (ECM). The ECM of most connective tissues is comprised predominantly of fiber forming collagens, with type I collagen representing the most abundant collagen type. As such, type I collagen-based materials have been developed with the goals of repairing acute tendon damage and promoting regeneration of native tendon tissue. There are several collagen-based scaffolds currently on the market for tendon repair such as Integra™, TenoGlide™, Zimmer™ Collagen Repair Patch and GRAFTJACKET™ Regenerative Tissue Matrix, which retain some of the hierarchical organization of collagen present in native tissues. However, a key feature that is present in the native type I-collagen ECM but is absent from most collagen-based biomaterials used for tissue repair and regeneration currently under development, is the hierarchical fibrillar structure of type I collagen that gives rise to its tensile strength and contributes to the adhesion, alignment, proliferation, and differentiation of cell types that reside in connective tissues such as tendons.

To promote collagen self-assembly into a form factor appropriate for tendon repair and tendon tissue engineering, a variety of collagen spinning approaches have been developed including wet spinning, and dry spinning. Wet spinning can produce filaments with diameters between 8 and 600 μm with throughputs ranging from 1 to 10 meters per hour. Wet spinning has been used previously to produce collagen multifilament yarns comprised of 6 single filaments each having a diameter of 80 μm. These wet spun multifilament yarns were subsequently crosslinked with glutaraldehyde and had similar characteristics to tendon collagen fibers in the wet state with a tensile strength of about 40 MPa. However, this approach was relatively low throughput and the collagen monofilaments obtained by the wet spinning process were irregular in terms of their cross-sectional geometry. To produce finer fibers on the order of single microns down to hundreds of nanometers in diameter, it is possible to electrospin the collagen, typically in the presence of a polymer. For example, electrospinning has been used to produce collagen/polylactic acid nanofibrous mats that could be rolled into bundles to recapitulate tendon fascicle structures. However, to date, these approaches are low throughput and result in either individual or loosely consolidated monofilaments, thus limiting applicability for tendon repair and tendon tissue engineering, both of which require relatively large quantities of high tensile strength material. In comparison, dry spinning of a polymer dissolved in a volatile solvent is an industrial-scale process that can produce monofilament at rates of tens of meters per second. However, the solvents used in most dry spinning processes have potential to denature proteins including collagen. Contact drawing is a dry spinning variant where the polymer is a water soluble polymer such as dextran or poly(ethylene oxide) (PEO) and the drawing process is initiated by a nucleation element, such as a pin, which is inserted into the solution and retracted at about 1 m/s, for example. Using contact drawing, aqueous collagen/polymer mixtures can be converted to composite filaments with diameters between 1 and 10 μm, for example, which are then processed into pure collagen fibers by eluting the polymer in a buffered aqueous solution. The resulting collagen fibers have been shown to support the alignment and growth of cells.

There remains a need for a commercially useful process to produce multifilament collagen fiber bundles that possess the hierarchical fibrillar structure of natural collagen (e.g., type I collagen).

### SUMMARY

A process for producing a multifilament collagen fiber bundle comprises treating a polymer-collagen fiber bundle comprising a plurality of monofilaments of collagen supported on a support polymer with a series of liquid media having a decreasing osmotic pressure to remove the support polymer from the polymer-collagen fiber bundle, the support polymer being more soluble than the collagen in the liquid media.

A process for producing a multifilament collagen fiber bundle comprises treating a polymer-collagen fiber bundle comprising a plurality of monofilaments of collagen supported on a support polymer with a series of liquid media having a decreasing concentration of the support polymer to remove the support polymer from the polymer-collagen fiber bundle, the support polymer being more soluble than the collagen in the liquid media.

A multifilament collagen fiber bundle comprises collagen fibers having diameters of at least 0.3 μm, each collagen fiber comprising collagen fibrils of smaller diameter than the collagen fibers, each collagen fibril comprising collagen microfibrils of smaller diameter than the collagen fibrils and each collagen microfibril comprising a bundle of collagen molecules, the collagen microfibrils forming a hierarchical D-band structure in the collagen fibrils, wherein adjacent collagen microfibrils are shifted by ⅙^{th} of the D-band in comparison to microfibrils in naturally occurring collagen of a same type.

A process for producing a crosslinked multifilament collagen fiber bundle comprises crosslinking the multifilament collagen fiber bundle described above with a chemical crosslinking agent or with exposure to light.

A crosslinked multifilament collagen fiber bundle comprises crosslinked collagen fibers having diameters of at least 0.3 μm, each collagen fiber comprising collagen fibrils of smaller diameter than the collagen fibers, each collagen fibril comprising collagen microfibrils of smaller diameter than the collagen fibrils and each collagen microfibril comprising a bundle of collagen molecules, the collagen microfibrils forming a hierarchical D-band structure in the collagen fibrils, wherein adjacent collagen microfibrils are shifted by ⅙^{th} of the D-band in comparison to microfibrils in naturally occurring collagen of a same type.

A process for producing a crosslinked multifilament collagen fiber bundle comprises irradiating the multifilament collagen fiber bundle described above with ultraviolet radiation at a total energy dose of 0.1 J/cm² or more.

A crosslinked multifilament collagen fiber bundle comprises collagen fibers having diameters of at least 0.3 μm, each collagen fiber comprising collagen fibrils of smaller diameter than the collagen fibers, each collagen fibril comprising collagen microfibrils of smaller diameter than the collagen fibrils and each collagen microfibril comprising a bundle of collagen molecules, the collagen microfibrils forming a hierarchical D-band structure in the collagen fibrils, wherein adjacent collagen microfibrils are shifted by ⅙^{th} of the D-band in comparison to microfibrils in naturally occurring collagen of a same type, and wherein phenylalanine residues on adjacent collagen molecules are covalently bonded to form crosslinks between adjacent collagen molecules.

A collagen fiber comprises a diameter of at least 0.3 μm and comprises collagen fibrils of smaller diameter than the collagen fibers, each collagen fibril comprising collagen microfibrils of smaller diameter than the collagen fibrils and each collagen microfibril comprising a bundle of collagen molecules, the collagen microfibrils forming a hierarchical D-band structure in the collagen fibrils, wherein adjacent collagen microfibrils are shifted by ⅙th of the D-band in comparison to microfibrils in naturally occurring collagen of a same type.

A collagen fiber comprises: atelomeric collagen microfibrils forming a hierarchical D-band structure in the collagen fibrils, wherein adjacent collagen microfibrils are shifted by ⅙th of the D-band in comparison to microfibrils in naturally occurring collagen of a same type; and, polyethylene oxide (PEO) in an amount of 60 wt % or less, based on total weight of the collagen fiber.

The multifilament collagen fiber bundles and the collagen fibers are useful as collagen-based biomaterials, especially for tissue repair and regeneration, especially for connective tissues, such as tendons. Thus, the multifilament collagen fiber bundles and the collagen fibers are useful in collagen-based sutures, as a scaffold for tendon repair, and in regenerative medicine as a scaffold to support the growth and differentiation of cells, for example tenogenic stem cells.

Further features will be described or will become apparent in the course of the following detailed description. It should be understood that each feature described herein may be utilized in any combination with any one or more of the other described features, and that each feature does not necessarily rely on the presence of another feature except where evident to one of skill in the art.

### BRIEF DESCRIPTION OF THE DRAWINGS

For clearer understanding, preferred embodiments will now be described in detail by way of example, with reference to the accompanying drawings, in which:

FIG. 1 depicts a fabrication process for PEO-collagen multifilament bundles. (A) Pulling of the PEO-collagen monofilaments on a frame after contacting between the PEO-collagen solution and the pin array, gathering and twisting of monofilaments from both ends, and rolling of twisted monofilaments to obtain a compact multifilament. (B) Representative image of rolled multifilaments with a length of about 9 cm. (C) Representative images of rolled multifilaments with tunable diameter. (D) Epifluorescence images of PEO-collagen-FITC-PEG/PEO-Phalloidin-TRITCR multifilament bundles demonstrating tilt with respect to the multifilament axis.

FIG. 2 depicts optical microscopy images of the washing process of a PEO-collagen multifilament. (A) Multifilaments immersed directly in 1×PBS after fabrication start to disaggregate immediately. (B) Washing in a graded series of PEO: 1×PBS.

FIG. 3 depicts Wide Angle X-Ray Scattering (WAXS) patterns for a (A) PEO multifilament, (B) PEO-collagen multifilament, (C) washed PEO-collagen multifilament with PEO: 1×PBS solutions, (D) washed PEO-collagen multifilament with PEO: 10×PBS solutions, and (E) rat tail tendon. Arrow indicates the multifilament direction. (F) Azimuthally averaged WAXS profiles.

FIG. 4 depicts Small Angle X-Ray Scattering (SAXS) patterns for a (A) PEO-collagen multifilament, (B) washed PEO-collagen multifilament in PEO: 1×PBS, (C) washed PEO-collagen multifilament in PEO: 10×PBS, and (D) rat tail tendon. Arrow indicates the multifilament direction. (E) SAXS profiles.

FIG. 5 depicts scanning electron micrographs (SEM) of PEO-collagen multifilament bundles washed with PEO: 10×PBS. (A) Magnification 2000×. (B) Magnification 50,000X. (C) Zoomed in panel in (B). (D) Profile extracted between the two arrowheads in (C).

FIG. 6 depicts transmission electron micrographs (TEM) of PEO-collagen monofilaments washed with (A) 1×PBS and (B-C) PEO: 10×PBS solutions.

FIG. 7 depicts a collagen fibril model. (A) Model based on the microfibrillar structure of type I collagen and (B) corresponding electron density profile along a microfibril (C). Collagen fibril model where each microfibril is staggered by one-sixth of the D-band in (A) and corresponding electron density profile (D) constructed using the density profile in (C).

FIG. 8 depicts A) Stress-strain curves in wet state. (B) ultimate tensile strength (UTS), (C) Young's modulus and (D) swelling ratio for PEO-collagen multifilament bundles washed with PEO: 10×PBS solutions, without crosslinking and after crosslinking in the dry state with 0.4 J/cm² UVC, 0.8 J/cm² UVC, 2 J/cm² UVC, 10 J/cm² UVC, and 1% glutaraldehyde after washing. Error bars represent standard deviation. Dashed lines in (B) and (C) indicate UTS and Young's modulus of rat tail, respectively (Y. P. Kato, D. L. Christiansen, R. A. Hahn, S. J. Shieh, J. D. Goldstein, F. H. Silver, Biomaterials 1989, 10, 38).

FIG. 9 depicts WAXS patterns for (A) washed PEO-collagen multifilaments in graded PEO: 10×PBS solutions and (B) washed PEO-collagen multifilaments in graded PEO: 10×PBS solutions after 10 J/cm² UVC. (C) WAXS profiles. Arrow indicates the multifilament direction. (D) ATR-FTIR spectra.

FIG. 10 depicts graphs showing (A) thermogravimetric (TG) and (B) differential thermogravimetric (DTG) curves of PEO-collagen multifilaments and PEO-collagen multifilaments after washing in a graded series of PEO: 10×PBS.

FIG. 11 depicts optical images of more complex 3D braided structures formed by braiding three PEO-collagen multifilaments.

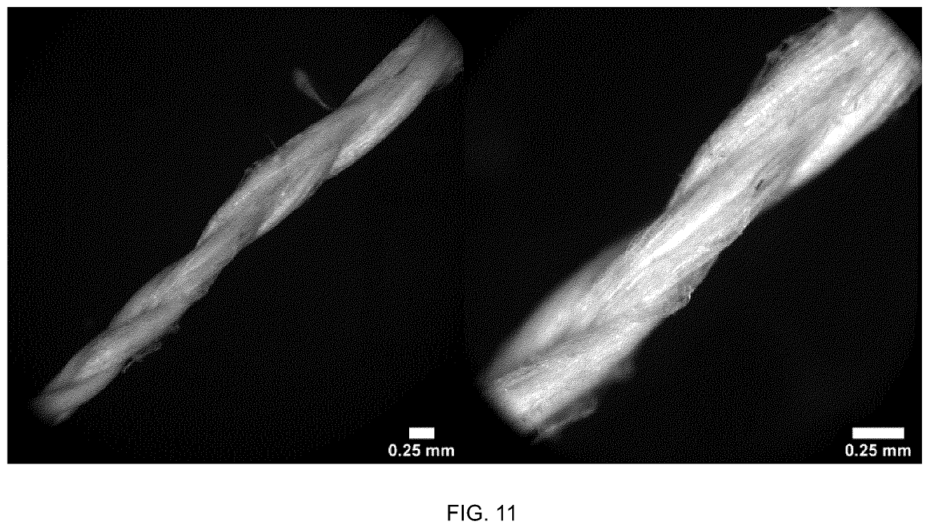

### DETAILED DESCRIPTION

Multifilament collagen fiber bundles are produced from polymer-collagen fiber bundles. The polymer-collagen fiber bundles comprise monofilaments of collagen supported on a support polymer. Removal, i.e., extraction, of the support polymer from the collagen to provide multifilament collagen fiber bundles may be accomplished by selectively removing the support polymer to leave behind the collagen. In some embodiments, selective removal of the support polymer may be accomplished by a liquid medium in which the support polymer is more soluble than the collagen, the liquid medium selectively dissolving the support polymer leaving the collagen undissolved. The collagen is preferably insoluble in the liquid medium, for example at a level of less than about 0.01 mg collagen per mL of the liquid medium.

However, it has been found that removing the support polymer all at once prevents the collagen from self-assembling into physically integral collagen fibers. To overcome this problem, it has now been found that subjecting the polymer-collagen fiber bundles to successive support polymer removal steps involving liquid media having successively decreasing concentrations of the support polymer provides for a gradual removal of the support polymer from the polymer-collagen fiber bundles giving sufficient time for the collagen to self-assemble into physically integral multifilament collagen fiber bundles. Further, the multifilament collagen fiber bundles produced by the process are hierarchically structured whereby the average axial periodicity in the bulk of the multifilament collagen fiber bundle exhibits a D-band structure very similar to that of native collagen except for a shift of the positions of amino acids relative to positions of the amino acid in adjacent collagen molecules across an axial span of the D-band structure. The shifted D-band structure statistically brings more phenylalanine units into alignment between adjacent collagen molecules permitting the use of photo-crosslinking (e.g., ultraviolet C crosslinking) to crosslink collagen molecules across phenylalanine units without the need for a photo-initiator or a chemical crosslinker while being able to provide, in some embodiments, a crosslinked multifilament collagen fiber bundle that has tensile properties that meet requirements for tendon replacement (i.e., Young's modulus in a range of 450-2,000 MPa and ultimate tensile strength (UTS) in a range of 25-148 MPa).

Polymer-collagen fibers in the polymer-collagen fiber bundles may be produced by any suitable fiber forming process such as contact drawing and other dry spinning approaches, wet spinning, electrospinning and the like. In some embodiments, contact drawing (e.g., multi-pin contact drawing) from a pre-strand composition containing the support polymer and collagen is used to generate a multitude of polymer-collagen monofilaments (fibers). The polymer-collagen monofilaments may be of any length. In some embodiments, the polymer-collagen monofilaments have lengths of up to 50 cm, for example 0.5-20 cm. The polymer-collagen monofilaments have diameters of at least 0.3 μm. In some embodiments, the diameter is at least 0.5 μm or at least 1 μm. In some embodiments, the diameter is in a range of 0.3-50 μm. The process is easily scalable and can produce thousands of aligned polymer-collagen monofilaments under ambient conditions without the need for specialized equipment or hazardous materials. The polymer-collagen monofilaments are then consolidated into tightly packed polymer-collagen fiber bundles, for example through twisting and rolling.

As stated above, in some embodiments the process of producing multifilament collagen fiber bundles comprises subjecting polymer-collagen fiber bundles to a series of liquid media having a decreasing concentration of the support polymer. The series may comprise a continuous reduction in the concentration of the support polymer in which the liquid medium is continuously replaced or diluted with medium having less and less support polymer, a discontinuous process involving a decrease in the concentration of the support polymer at certain time intervals or a combination thereof. Whether in a continuous or discontinuous process, successive treatment steps (i.e., wash steps) are used involving liquid media having successively decreasing concentrations of the support polymer. The decreasing concentration may be realized by entirely replacing the liquid medium in successive steps, each successive liquid medium batch having a lower concentration of the support polymer. Alternatively, or in addition, the decreasing concentration may be realized by continuous or time-interval dilution of the liquid medium without entirely replacing the liquid medium at each step. A combination of successive dilution and replacement may be utilized. The polymer-collagen fiber bundles are subjected to at least two treatment steps whereby the concentration of the support polymer is less in the final treatment step than in the initial treatment step. Two, three, four, five, six or more successive steps may be utilized.

The initial concentration of the support polymer in the liquid medium depends on the support polymer. However, an initial concentration of 10 wt % or less, based on total weight of the liquid medium, is generally suitable. In some embodiments, a concentration of 0.25-5 wt % is used. In some embodiments, a concentration of 0.75-2 wt % is used.

Successive concentrations of the support polymer in the liquid media are lower than the initial concentration. For example, a second treatment step may comprise a liquid medium having a concentration of support polymer in a range of 0-0.75 wt %, based on total weight of the liquid medium in the second treatment step. In some embodiments, a concentration of 0.35-0.75 wt % is used in the second treatment step. A third treatment step, when utilized, may comprise a liquid medium having a concentration of support polymer in a range of 0-0.35 wt %, based on total weight of the liquid medium in the second treatment step. In some embodiments, a concentration of 0.1-0.35 wt % is used in the second treatment step. In a continuous process, the concentrations for the support polymer in successive steps can be points along a continuously decreasing concentration. In some embodiments, successive treatment steps involve successively halving the concentration of the support polymer in each successive treatment step. Irrespective of the number of treatment steps utilized or whether a continuous reduction in support polymer concentration is employed, a final treatment step may involve a liquid medium having some support polymer (i.e., a concentration of support polymer greater than 0 wt %) or an absence of support polymer (i.e., a concentration of support polymer of 0 wt %).

In some embodiments, each treatment step is conducted for a length of time that results in the slow removal of the support polymer from the polymer-collagen fiber bundles until the concentration of the support polymer in the liquid medium reaches an undesirable level. The length of time for each step depends, in part, on the solubility of the support polymer in the liquid medium used in the step. Generally, the length of time for each step is in a range of 1-24 hours. The earlier treatment steps having greater concentrations of the support polymer, and which result in removing larger amounts of the support polymer from the polymer-collagen fiber bundles, are generally conducted for a shorter length of time than later treatment steps that have lesser concentrations of the support polymer and where the polymer-collagen fiber bundles have less of the support polymer to remove. In some embodiments, the earlier treatment steps are conducted for a length of time in a range of 1-8 hours, for example 2-6 hours, while the later treatment steps are conducted for 3-24 hours, for example 8-18 hours. The above time ranges are general and some later treatment steps may be conducted for shorter lengths of times while some earlier treatment steps may be conducted for longer periods of time.

Since collagen is insoluble in water, the liquid media may comprise aqueous media in which the collagen is insoluble or sparingly soluble, but in which the support polymer is soluble or at least dissolves faster than the collagen. With aqueous media, the treating of the polymer-collagen fiber bundles comprises hydrating the polymer-collagen fiber bundles. In some embodiments, one or more of the aqueous media comprises a buffer. In some embodiments, one or more of the aqueous media may comprise unbuffered water. In some embodiments, one or more of the aqueous media may comprise a biologically useful medium such as a cell culture medium, an alcohol/water mixture, and the like. Buffers include, for example, phosphate-buffered saline (PBS), TRIS, HEPES, PIPES, MES, MOPS, Imidazole or mixtures thereof.

The process of removing the support polymer may be conducted at any suitable temperature that does not denature the collagen and does not freeze the liquid media. In some embodiments, the temperature at which the process is conducted is in a range of 20-37° C.

Collagen is the main structural protein in the extracellular matrix found in various connective tissues (e.g., cartilage, bones, tendons, ligaments, and skin) of mammals. Some examples of collagen are type I (skin, tendon, vasculature, organs, bone), type II (cartilage), type III (reticulate), type IV (basal lamina, the epithelium-secreted layer of the basement membrane) and type V (cell surfaces, hair, and placenta). In some embodiments, the collagen comprises type I collagen. In some embodiments, the collagen is atelomeric. Atelomeric collagen has telomeric peptides removed, for example by pepsin solubilization. Collagen is a linear polymer having a diameter of about 1 nm. A plurality of collagen molecules, for example five collagen molecules can bundle adjacent to each other to form a collagen microfibril. A microfibril has a diameter in a range of 4-5 nm. A plurality of microfibrils, for example seven microfibrils, can bundle adjacent to each other to form a fibril. A fibril has a diameter in a range of 10-100 nm, for example 30-50 nm. A plurality of fibrils can bundle adjacent to each other to form a fiber. Collagen fibers produced in the present process may have diameters of at least 0.3 μm. In some embodiment, the diameter is at least 0.5 μm or at least 1 μm. In some embodiments, the diameter is in a range of 0.3-50 μm, for example 1-10 μm.

The support polymer provides a scaffold on which the collagen can assemble to maintain stability of the collagen strand during strand formation. Some examples of support polymers include dextran, polyethylene oxide (PEO), polyvinyl acetate (PVA), polyethylene glycol (PEG), hydroxypropyl cellulose, poly(2-ethyl-2-oxazoline), poly(4-styrenesulfonic acid-co-maleic acid), poly(acrylic acid), poly(diallyldimethyl ammonium chloride), poly(methacrylic acid), poly(methyl vinyl ether-alt-maleic acid), poly(vinylpyrrolidone) (PVP), low-density polyethylene (LDPE), high-density polyethylene (HDPE), polypropylene (PP), polyvinyl chloride (PVC), polystyrene (PS), nylon, polytetrafluoroethylene, thermoplastic polyurethanes, crosslinked polymers thereof and copolymers thereof. In some embodiments, the support polymer comprises polyethylene oxide (PEO). In some embodiments, only one type of support polymer is utilized in the polymer-collagen fiber bundles, while in other embodiments, more than one type of support polymer is utilized in the polymer-collagen fiber bundles.

The support polymer may be present in the fibers. In some embodiments, the support polymer is present in the fibers in an amount of 60 wt % or less, based on total weight of the fiber. In some embodiments, the support polymer is present in an amount of 0.0001-50 wt %. In some embodiments, prior to being removed from the fibers, the support polymer is present in the fibers in an amount of 40-60 wt %, for example 47-53 wt %. In some embodiments, after being removed from the fibers, the support polymer is present in the fibers in an amount of less than 20 wt %, for example 0.0001-20 wt % or 0.0001-5 wt %.

The product of the process for producing a multifilament collagen fiber bundle comprises a multifilament collagen fiber bundle comprising collagen fibers having diameters of at least 0.3 μm. Each collagen fiber comprises collagen fibrils of smaller diameter than the collagen fibers. Each collagen fibril comprises collagen microfibrils of smaller diameter than the collagen fibrils. Each collagen microfibril comprises a bundle of collagen molecules. The collagen microfibrils form a hierarchical D-band structure in the collagen fibrils. Adjacent collagen microfibrils are shifted by ⅙^{th} of the D-band in comparison to microfibrils in naturally occurring collagen of a same type.

The multifilament collagen fiber bundles produced by the process are not exactly the same as native collagen. The multifilament collagen fiber bundles have banding structures (D-bands), but the D-band structure differs from native collagen. If the D-band is modeled as vertical slices through horizontally oriented layers of collagen molecules in the bundle, there is a missing collagen molecule in comparison to native collagen at ⅙th the distance (about 11 nm). By taking angled slices through the bundled layers of collagen molecules, the D-band can be defined as having a missing collagen molecule every 67 nm.

It has been found that an additive added during fiber formation is not extracted by the liquid media in the process. Therefore, it is possible to include one or more additives in the multifilament collagen fiber bundle by introducing the one or more additives into the polymer-collagen fibers during fiber formation. In some embodiments, when contact drawing is used to produce the fibers, the one or more additives may be included in the pre-strand composition and become entrained in the polymer-collagen fibers as the fibers are drawn from the pre-strand composition. In some embodiments, the one or more additives comprises functional additives commonly used in biochemistry, for example in cell culture. In some embodiments, the functional additives include saccharides, growth factors, hormones, extracellular matrix proteins (ECM) (e.g., fibronectin, laminin, etc.), enzymes, cytokines, chemokines, antibodies (e.g., monoclonal antibodies), anti-inflammatories, steroids, immune suppressants, chemotherapy agents, lipids, hyaluronic acid, liposomes, micro/nano capsules, genetic materials (e.g., DNA (e.g., plasmids), RNA (e.g., mRNA, interfering RNA), nucleotides), amino acids, extracellular vesicles, whole cells, metallic ions, non-metallic ions, nanoparticles (e.g., carbon nanotubes, metallic nanoparticles), solid microparticles (e.g., metal, plastics, glass, etc.), colorants, surfactants, detergents, vitamins, bases, mineral and organic acids (e.g., citric acid), other natural health products, other small molecule pharmaceuticals (e.g., minocycline, riluzole, dalfampridine, escitalopram, deoxygedunin, 7,8-dihydroxflavone, quercetin, dexamethasone, tacrolimus) and combinations thereof. The functional additives are preferably pharmaceutically active agents, precursors of pharmaceutically active agents or combinations thereof.

The multifilament collagen fiber bundles may be crosslinked to produce crosslinked multifilament collagen fiber bundles. The crosslinking may be accomplished by contacting the multifilament collagen fiber bundles with a chemical crosslinking agent (e.g., glutaraldehyde, carbodiimides, genipin, transglutaminase, lysyl oxidase, other enzymes, and the like), by physical crosslinking with dehydrothermal treatment or by photo-crosslinking with light of a suitable wavelength (e.g., ultraviolet (UV) light with wavelengths in a range of 100-400 nm, especially UVC with wavelengths in a range of 100-280 nm).

Because the shifted D-band structure of the multifilament collagen fiber bundles statistically brings more phenylalanine units into register between adjacent collagen molecules, it is possible to use photo-crosslinking to crosslink collagen molecules across phenylalanine units without the need for a photo-initiator or a chemical crosslinker while being able, in some embodiments, to provide a crosslinked multifilament collagen fiber bundle that has improved mechanical properties and/or to tune the mechanical properties of the crosslinked multifilament collagen fiber bundle by tuning the total light energy dose. The shifted D-band structure permits the use of higher total energy doses than is possible with native collagen and leads to increased crosslinking density when photo-crosslinking is employed, compared to photo-crosslinking of native collagen. In some embodiments, the crosslinking density in the crosslinked multifilament collagen fiber is in a range of 1 million to 26 million crosslinks per μm³. In some embodiments, the crosslinking density is in a range of 2 million to 26 million crosslinks per μm³. In some embodiments, the crosslinking density is in a range of 3.5 million to 26 million crosslinks per μm³. 5.5 million to 26 million crosslinks per μm³. 7 million to 26 million crosslinks per μm³.

Thus, in some embodiments, a process for producing a crosslinked multifilament collagen fiber bundle comprises irradiating a multifilament collagen fiber bundle with ultraviolet radiation at a total energy dose of 0.1 J/cm² or more, the total energy dose is in a range of 0.1-100 J/cm². In some embodiments, the total energy dose is in a range of 5-100 J/cm². In some embodiments, the total energy dose is in a range of 5-50 J/cm². Photo-crosslinking is preferably done when the multifilament collagen fiber bundles are dry. UVC radiation at a wavelength in a range of 250-260 nm (e.g., 254 nm) is well suited for photo-crosslinking adjacent phenylalanine units. While the photo-crosslinking can be performed with or without a photo-initiator, the process permits photo-crosslinking in an absence of a photo-initiator while still resulting in a crosslinked multifilament collagen fiber bundle having high crosslinking density and good mechanical properties.

The crosslinked multifilament collagen fiber bundles retain the D-band structure of the multifilament collagen fiber bundles and phenylalanine residues on adjacent collagen molecules are covalently bonded to form crosslinks between the adjacent collagen molecules.

In some embodiments where photo-crosslinking is employed, the crosslinked multifilament collagen fiber bundles have an ultimate tensile strength of 1 MPa or greater and/or a Young's modulus of 20 MPa or greater. In some embodiments, the ultimate tensile strength and/or the Young's modulus can even exceed that of multifilament collagen fiber bundles crosslinked with a chemical crosslinker. When photo-crosslinked at higher total energy doses (e.g., 5 J/cm² or higher, especially 10 J/cm² or higher), the crosslinked multifilament collagen fiber bundles approach tendon-like mechanical performance. In some embodiments, the ultimate tensile strength is up to 250 MPa. In some embodiments, the ultimate tensile strength is in a range of 10-200 MPa. In some embodiments, the ultimate tensile strength is in a range of 25-150 MPa. In some embodiments, the Young's modulus is up to 3,000 MPa. In some embodiments, the Young's modulus is in a range of 50-2,500 MPa. In some embodiments, the Young's modulus is in a range of 450-2,000 MPa.

In the collagen molecule, there is a phenylalanine residue that repeats on a D/6 length-scale, where d is the length scale of collagen's characteristic density-banding, about 67 nm. When two phenylalanine residues on adjacent collagen molecules are in close proximity, they can be crosslinked with UVC. In the present crosslinking process, the combination of contact drawing polymer-collagen fibers and the treatment process that extracts the support polymer creates collagen fibers that, when exposed to UVC, still results in collagen fibers with a prominent order and density banding of D/6. Thus, the present process to produce multifilament collagen fiber bundles aligns the collagen molecules so that the phenylalanine residues are better aligned to allow for photo-crosslinking. Further, the photo-crosslinking process is highly tunable as increasing UV total energy dose increases the degree of crosslinking which increases the strength and stiffness of the fibers, which differs from what was known about collagen as the literature shows clear limits on physical crosslinking of collagen that are exceeded by the present process and product, all without the need of a photo-initiator.

### EXAMPLES

### Experimental Procedures

### Preparation of the PEO-Collagen Solution (Pre-Strand Composition)

PEO powder (8 MDa; Sigma Aldrich) was dissolved in a solution of type I collagen (about 6 mg/mL in 0.01 M HCl; Collagen Solutions) to achieve a PEO-collagen with a weight ratio of 1:1. The type I collagen utilized was a pepsin solubilized collagen, which removed telomeric peptides so that the collagen is atelomeric. By stirring with a metal spatula, small PEO clumps were dispersed as much as possible and then transferred to two syringes connected by a dual lock syringe tip. The PEO-collagen solution was transferred through the syringe tip between the two syringes at least 100 times to ensure homogenous dissolution of the PEO. The PEO-collagen solution was then stored at 4° C. for air bubbles to dissipate before being transferred to a −20° C. freezer. The frozen PEO-collagen solution was then lyophilized at −80° C. using a FD8508 lyophilizer (IIShinBioBase). The lyophilized PEO-collagen sponges were then chopped with tweezers and dissolved in 20 mM acetic acid (diluted from glacial acetic acid, ASC grade, VWR). To ensure the complete dissolution of the PEO and collagen, the mixture was again stirred with a metal spatula and transferred between two syringes as described above. The resulting PEO-collagen solution was stored at 4° C. overnight and then brought to ambient temperature for contact drawing. The concentration of the lyophilized PEO-collagen in acetic acid was 6.75 wt %.

### Collagen Multifilament Assembly, Hydration and Crosslinking

Contact drawing of PEO-collagen fibers was performed as previously described using a multi-pin array designed to nucleate fibers and a substrate tool designed to hold the viscous PEO-collagen solution (see WO 2022/032387 published Feb. 17, 2022, the entire contents of which is herein incorporated by reference.). Briefly, the PEO-collagen solution was spread on the flat rectangular substrate tool. A 30×55 array of cylindrical pins with a diameter of 0.6 mm, height of 5 mm, and center-to-center spacing of 1.6 mm was then used to produce a multitude of PEO-collagen monofilaments by contacting the pin array with the viscous solution and then pulling the pin array away from the rectangular plate. The drawing process was repeated for 30 cycles. Monofilament were collected over a frame, before being gathered by their ends, twisted by hand 10 times, and rolled into a tightly consolidated multifilament bundle. The imparted twist was visualized by epifluorescence imaging of multifilament bundles formed from solution of PEO-collagen/FITC-PEG (0.05 wt. %; MW 10K g/mol, Creative PEGWorks) and PEO/Phalloidin-TRITC (0.05 wt. %, MW 1231.40 g/mol, Sigma-Aldrich) using a Nikon Eclipse Ti optical microscope (Nikon Instruments). Two sets of pin arrays and rectangular plates were used to form monofilaments from each of these solutions. The fluorophore-labeled multifilaments were created by one elongation event with PEO-collagen/FITC-PEG followed by one elongation event with PEO/Phalloidin-TRITC. This process was repeated 10 times for a total of 20 fiber elongation events. All processes were carried out at 27±2° C. and relative humidity of 28±2%.

Following fiber consolidation, the PEO-collagen multifilament bundles were mounted on a custom 3D-printed stand and exposed to hydration in a graded series of either PEO: 1×PBS or PEO: 10×PBS. The hydration protocol was as follows: 1 wt. % PEO:PBS for 4 hrs; 0.5 wt. % PEO:PBS for 4 hrs; 0.25 wt. % PEO:PBS for 16 hrs; 100% PBS for 8 hrs; 100% PBS for 16 hrs; and water for 3 hrs). The dried collagen multifilament bundles were then either immersed for 1.5 hrs at room temperature in a crosslinking solution of 1.0 wt % glutaraldehyde or exposed to UVC radiation (UVP crosslinker CL-3000, Analytik Jena). UVC (wavelength 254 nm) was applied at total energies of 0.4 J/cm², 0.8 J/cm², 2 J/cm², and 10 J/cm².

### Wide- and Small-Angle X-Ray Scattering (WAXS and SAXS)

WAXS and SAXS patterns were acquired for pure PEO, PEO-collagen, PEO-collagen multifilaments after hydration, and for rat tail tendons. The WAXS patterns were acquired using a D8 Advance X-ray diffractometer (Bruker Co.) operating at a wavelength of 0.15406 nm. Data were recorded in the range (20) of 0-50° at a sample to detector distance of 70 mm with an exposure time of 600 s. The WAXS frames were integrated from a two-dimensional image into a one-dimensional powder pattern by azimuthal averaging using the Fit2D software package developed at the European Synchrotron Radiation Facility (ESRF). All multifilaments were mounted vertically in front of the instrument. SAXS measurements were performed on a SAXSpoint 2.0 (Anton Paar) equipped with a copper source, using an Eiger detector set at 575.6533 mm from the sample. The X-ray exposure time was 30 min per frame for a total of 4 frames. The samples were oriented perpendicular to the X-ray beam. FIT2D software was used to convert two-dimensional images into one-dimensional meridional profiles.

### Scanning Electron Microscopy (SEM)

The surface morphology of the collagen multifilament bundles was characterized by scanning electron microscopy using a Sigma 300 VP Field Emission SEM (Zeiss). Prior to imaging, the multifilaments were fixed with 2.5% glutaraldehyde in 1×PBS for 2 hrs and then rinsed with 1×PBS (3×10 min). Next, the multifilaments were rinsed with distilled water. The multifilaments were then dehydrated in a graded series of ethanol (50% ethanol for 1×10 min; 70% ethanol for 2×10 min; 95% ethanol for 2×10 min; 100% ethanol for 2×10 min, 100% dried for 1×10 min). The samples were then critical point dried using an EM CPD300 system (Leica Microsystems). The samples were then removed from the critical point dryer and were attached to SEM stubs using carbon tape, and an ultra-thin gold/palladium (80/20) layer was applied with a sputter coater EM ACE600 (Leica Microsystems). The multifilaments were observed at an accelerating voltage of 5 kV and a working distance between 7.5 and 11.5 mm. To study the collagen fibril structure within the monofilaments, high magnification scans (i.e., 50,000×) were used. Lower magnification (i.e., 2,000×) scans were used to observe the alignment of the monofilaments.

### Transmission Electron Microscopy (TEM)

PEO-collagen monofilaments were captured on 400 mesh copper grids coated using a formvar carbon film (Electron Microscopy Sciences). The monofilaments were either washed on the grid with a drop of 1×PBS for 2 min followed by 3 drops of water, 1 min each, or with graded PEO: 10×PBS solutions (i.e., 1 wt. % PEO:PBS for 1 hr; 0.5 wt. % PEO:PBS for 1 hr; 0.25 wt. % PEO:PBS for 1 hr; 100% PBS for 2×1 hr) followed by 1 drop of water for 5 sec, 1 drop of 1% glutaraldehyde in water for 1 min, and 3 drops of water for 5 sec each. In both cases the grids were stained with 2% uranyl acetate solution for up to 30 sec. The grids were then examined with a JEM-1230 transmission electron microscope (JEOL) equipped with a Hamamatsu ORCA-HR digital camera at an accelerating voltage of 80 kV.

### Mechanical Characterization

The mechanical properties including UTS and Young's modulus for collagen multifilament bundles were evaluated using a Mark-10 F105 tensile tester (Mark-10 Corporation) equipped with 0.5 N and 10 N force sensors, at a crosshead speed of 6 mm/min and a gauge length of 10 mm. The multifilament bundles were mounted on a custom-made 3D-printed frame and secured at both ends using Superglue (Elmer Products). Using a 4× objective lens on the Nikon Eclipse Ti microscope, brightfield images of the multifilament bundles were captured. ImageJ was used to measure the multifilament bundle diameters to calculate the cross-sectional areas to determine the tensile stress. The mounted multifilament was incubated in water for 1.5 hrs before diameter measurements were taken.

### Swelling Analysis

To quantify multifilament swelling, 5 multifilaments of 10 mm length were selected. The dry multifilament diameter was measured from images captured using the Nikon Eclipse Ti microscope equipped with a 10× objective lens. After hydrating the multifilament for 1.5 hr in water, the diameters were measured again. The swelling percent was calculated according to Equation 1.

$$Swelling Percent = \frac{{( Wet diameter )}^{2} - {( Dry diameter )}^{2}}{{( Dry diameter )}^{2}} \times 100 \qquad ( 1 )$$

### Attenuated Total Reflectance Fourier-Transform Infrared Spectroscopic Analysis (ATR-FTIR)

ATR-FTIR analysis was performed using a Nicolet iZ10 MX integrated FTIR microscope (Thermo Fisher Scientific) to record the vibrational modes of functional groups attributed to hydrated PEO-collagen multifilament. Multifilament bundles of 1 cm in length were placed on the ATR slide and fixed with adhesive tape at both ends. The spectra for each sample were recorded in ATR mode using a Slide-On ATR objective with a conical germanium crystal. All measurements were acquired in the 700-4000 cm⁻¹ range with a resolution of 8 cm⁻¹ and an aperture of 100×100 μm.

### Thermogravimetric Analysis (TGA)

The thermal stability of a PEO-collagen multifilaments and washed PEO-collagen multifilaments with PEO: 10×PBS solutions was analyzed up to 600° C. with a heating rate of 10° C./min under argon. Changes in weight percentage and the decomposition temperatures of the multifilaments were recorded with an SDT-Q600 (TA instrument) thermogravimetric analyzer.

### Results and Discussion:

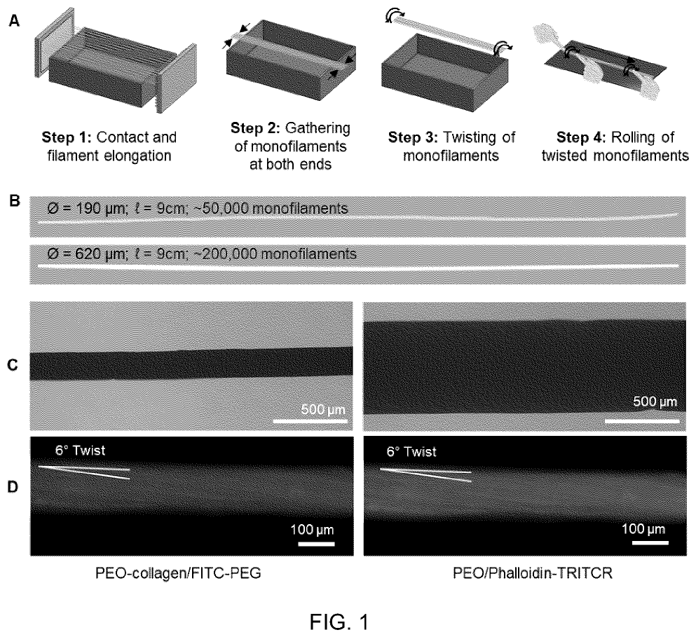

Using the contact drawing approach with a multi-pin tool and a flat plate to contain the viscous polymer-collagen solution, it was possible to produce thousands of aligned PEO-collagen monofilaments during each about 1-second-long contact-elongation event (FIG. 1, Panel A, Step 1). Following each contact event, a series of liquid bridges form from each of the pins which subsequently elongate into monofilaments by extensional flow. These monofilaments dry as they are collected over a frame. Unlike other approaches for fiber formation which may require the use of nozzles, high voltage power supplies, flow-rate control, and complex mechanical control, the only requirements for fiber formation by contact drawing are that the polymer chains form entanglements in solution, that the pull speed is sufficiently fast to avoid relaxation of the entanglements, and that the pins on the array are spaced sufficiently far apart to prevent the liquid bridges from fusing together during fiber elongation.

### Production of Collagen Multifilament Bundles

Thirty contact-elongation cycles were required to produce about 50,000 monofilaments over a 12×8 cm frame. The monofilaments were consolidated by gathering them together at their ends and simultaneously twisting clockwise and counter-clockwise from both ends (FIG. 1, Panel A, Steps 2 and 3), before gently rolling the twisted multifilament on a flat plastic surface (FIG. 1, Panel A, Step 4). This process resulted in well consolidated multifilament structures of at least 9 cm in length, with diameter tunable by varying the number of monofilaments used to form each bundle (FIG. 1, Panel B and FIG. 1, Panel C). For a typical multifilament bundle formed from 50,000 monofilaments, the average diameter was 190 μm, with a twist angle of 6°+2 (FIG. 1, Panel D).

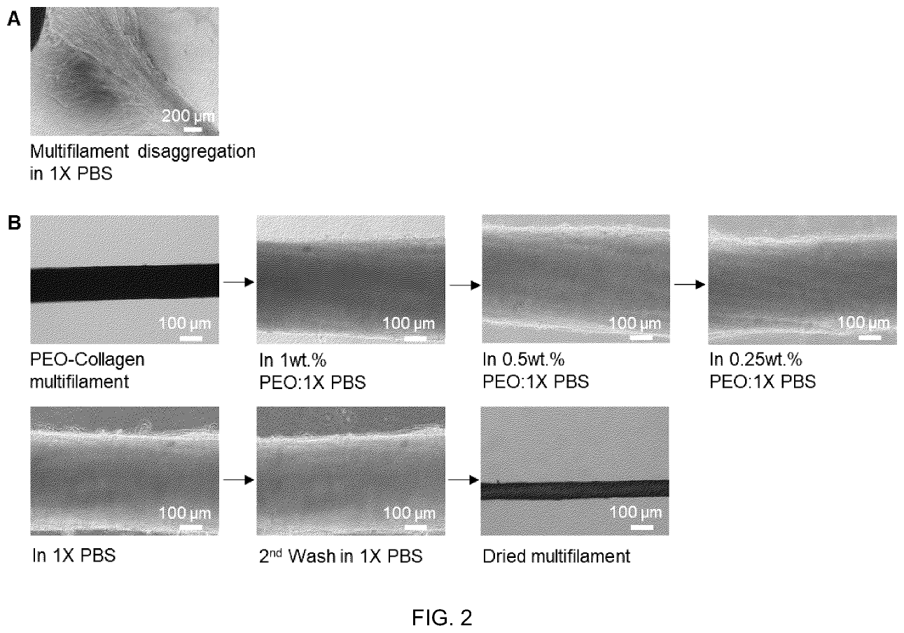

If the multifilament bundle is immersed directly in 1×PBS, the monofilaments break and disaggregate (FIG. 2, Panel A). One possible explanation for this result is that immersing the multifilament directly into PBS generates an osmotic-shock due to the large difference in PEO concentration between the multifilament and the surrounding PBS. Thus, to promote the self-assembly of collagen, remove the PEO, and preserve multifilament bundle structure, a hydration protocol was developed that involved washing the PEO-collagen multifilaments with decreasing concentrations of PEO in PBS. Washing the multifilament with decreasing concentrations of PEO in PBS decreases the osmotic shock at each washing step. Thus, the multifilament bundles were washed in a graded series of PEO buffered with either 1× or 10×PBS (FIG. 2, Panel B). The stepwise hydration approach preserves the twist angle of the multifilament bundle and provides enough time for collagen molecules to self-assemble into fibrils.

### Molecular Alignment and Packing of Collagen in Multifilament Bundles

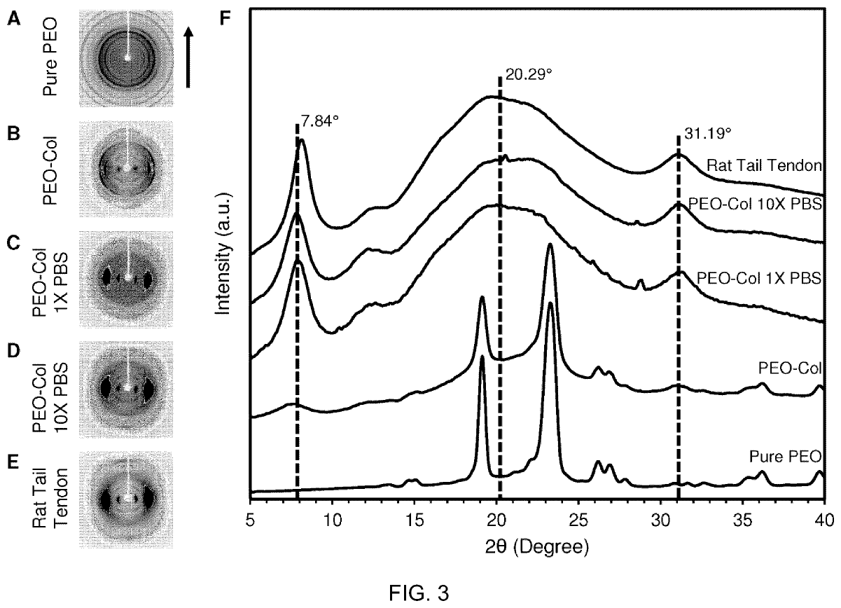

The azimuthally averaged WAXS profiles for pure PEO multifilament bundles (FIG. 3, Panel A), PEO-collagen multifilament bundles (FIG. 3, Panel B to FIG. 3, Panel D), and rat tail tendon (FIG. 3, Panel E) are presented in FIG. 3, Panel F. The WAXS pattern for pure PEO multifilament bundles indicates alignment of the PEO chains along the multifilament axis (FIG. 3, Panel A). The corresponding WAXS profile (FIG. 3, Panel F) has two high-intensity diffraction peaks at 19.14° and 23.27°, which can be assigned to the (120) and (112) planes of crystalline PEO, respectively. These sharp peaks arise from polyether chains being ordered as a result of van der Waals interactions between PEO chains. It shows that the multi-pin contact drawing technique and all fabrication processes used herein to prepare the collagen multifilament bundle did not affect the formation of PEO crystals. As can be seen in FIG. 3, Panel B, for the PEO-collagen multifilament bundle, two spots appear close to the beam center. The small peak centered at a 2θangle of 7.68° (d=1.151 nm) (FIG. 3, Panel F) shows the presence of collagen and is assigned to the intermolecular lateral packing of collagen triple helices. Adding collagen does not change the ordering of the PEO chains. Several new peaks appear after hydrating the PEO-collagen multifilament bundles in either a graded series of PEO: 1×PBS or PEO: 10×PBS to remove the PEO, indicating the presence and alignment of collagen triple helices along the multifilament direction (FIG. 3, Panel C and FIG. 3, Panel D; FIG. 3, Panel F). The peaks at (2θ=31.19°, d=0.287 nm) and (2θ=20.29°, d=0.437 nm) are assigned to the distance between residues along the triple helix axis and amorphous scattering originating from disordered portions of the collagen molecules, respectively. The WAXS patterns of the multifilament bundles after PEO hydration are indistinguishable from the well-aligned triple helices present in rat tail tendon (FIG. 3, Panel E and FIG. 3, Panel F). The main difference between collagen chains in the tendon and in the multifilament is a slight increase in the intermolecular lateral packing distance between collagen molecules (Tendon: 2θ=8.16°, d=1.08 nm; Multifilament: 2θ=7.84°, d=1.127 nm) (FIG. 3, Panel F).

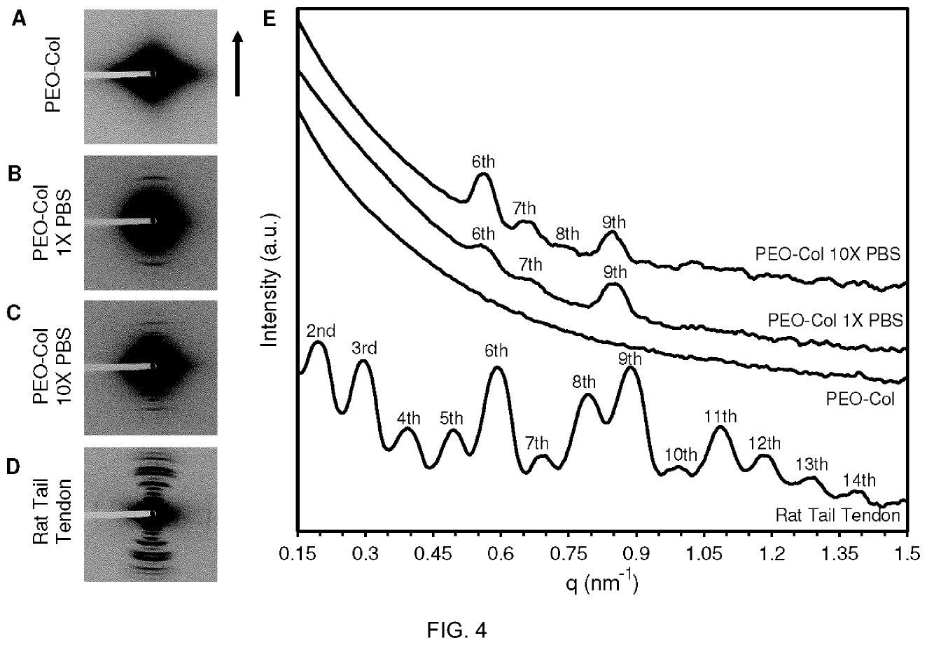

To gain a better understanding of the supramolecular structure of collagen molecules within the multifilament bundles, SAXS analysis was used. FIG. 4, Panel A to FIG. 4, Panel D depict SAXS patterns for a PEO-collagen multifilament, washed PEO-collagen multifilament with PEO: 1×PBS solutions, washed PEO-collagen multifilament with PEO: 10×PBS solutions, and rat tail. SAXS profiles for all these conditions are shown in FIG. 4, Panel E. The PEO-collagen multifilament did not exhibit a periodic SAXS pattern as demonstrated in FIG. 4, Panel A, which suggests that collagen molecules do not self-assemble into a tendon-like packing of collagen molecules during the fiber process because of the presence of PEO chains. When the PEO-collagen multifilament was washed in a graded series of either PEO: 1×PBS or PEO: 10×PBS, 4 arcs were present (FIG. 4, Panel B and FIG. 4, Panel C) with d-spacing of 11.2 nm, 9.65 nm, 8.47 nm, and 7.41 nm, respectively. No other arcs were observed at smaller angles as in the rat tail tendon sample (FIG. 4, Panel D). However, the observed arcs can be indexed as the 6th, 7th, 8th, and 9th order of a 67.2 nm D-band repeat. This is slightly longer than the D-band repeat measured for rat tail tendon of 64.8 nm. A possible explanation for this finding is that after washing, the multifilament contains collagen microfibrils with a 67.2 nm D-band repeat that are staggered by one-sixth of that repeat. Such an arrangement would produce collagen fibrils with a 11.2 nm axial periodicity. A similar structure was observed previously by electron microscopy in the case of type I collagen fibrils assembled at pH 7.

### Collagen Multifilament Architecture

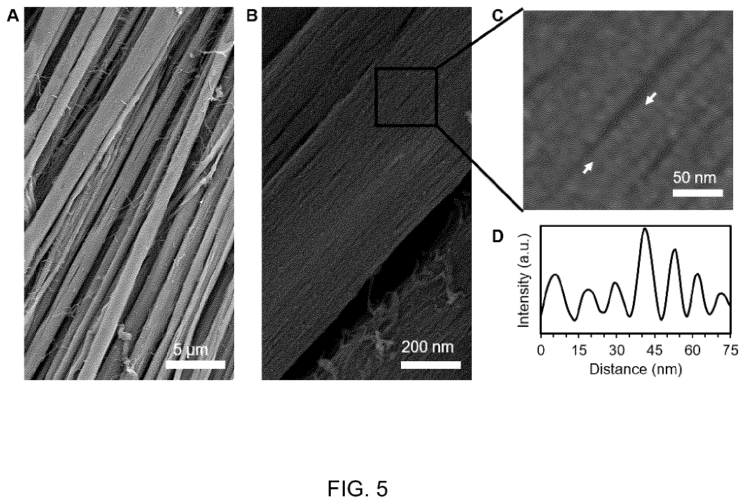

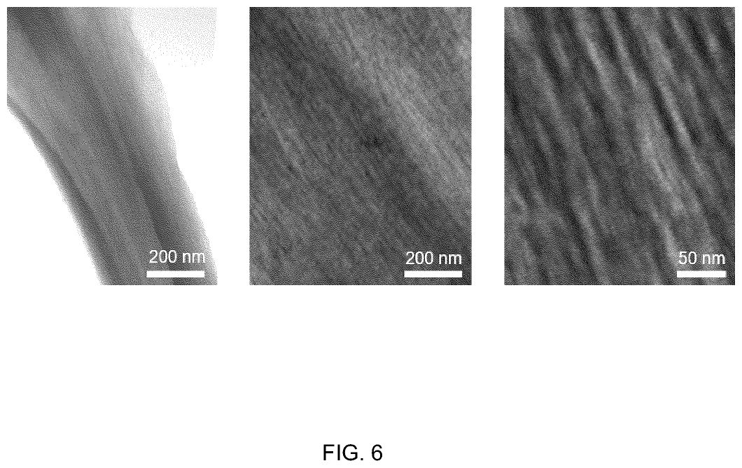

Preservation of the collagen multifilament architecture after hydration was confirmed by scanning electron microscopy (SEM) (FIG. 5, Panel A). Each collagen monofilament contains numerous closely packed fibrils of diameter 16±5 nm (n=222) (FIG. 5, Panel B). Each fibril displays a banded pattern (FIG. 5, Panel C) with a repeat of 10±3.8 nm (n=84), which agrees with the 11.2 nm axial periodicity observed by SAXS. The visibility of this spacing in the SEM images varied along the length of each collagen fibril, with short segments showing a clear periodicity (FIG. 5, Panel D). In comparison, banding was not visible by transmission electron microscopy (TEM) after a 2 min wash in 1×PBS (FIG. 6, Panel A). These rapidly washed collagen monofilaments displayed well-aligned collagen fibrils with no indication of axial repeat. This is consistent with the idea that lateral aggregation of the collagen molecules into microfibrils and fibrils occurs before the development of an axial repeat. After the graded wash with one hour per step, the collagen fibrils were still present and the appearance of a banding pattern was observed along the monofilament axis (FIG. 6, Panel B and FIG. 6, Panel C). However, the quality and length of the axial repeat varied from one monofilament to the next (FIG. 6, Panel B compared to FIG. 5, Panel C), with a few monofilaments showing an axial repeat in the 5-7 nm range (FIG. 6, Panel C) corresponding to a one-eleventh of the D-band repeat.

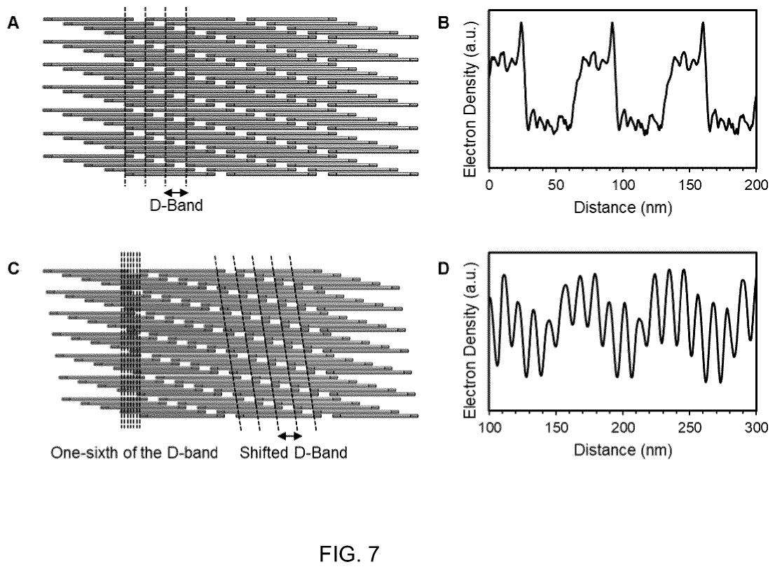

A model for the organization of the collagen fibrils within the collagen multifilament is presented in FIG. 7. The model is based on the microfibrillar structure of rat tail tendons obtained by Orgel et al. (J. P. R. O. Orgel, T. C. Irving, A. Miller, T. J. Wess, Proc. Natl. Acad. Sci. U.S.A. 2006, 103, 9001). A stick-model illustrating the expected quarter-stagger arrangement of the molecules for 6 parallel microfibrils is shown in FIG. 7, Panel A, along with the expected electron density along the microfibril axis (FIG. 7, Panel B)]. FIG. 7, Panel C illustrates the collagen fibril model where each microfibril is staggered by one-sixth of the D-band repeat and the corresponding electron density profile is constructed using the density profile of a quarter-staggered microfibril (FIG. 7, Panel D). As a result of the inter-microfibril staggering, the D-band repeat (67 nm) is almost completely removed and replaced by a prominent (11 nm) repeat. It should be noted that the electron density fluctuations are mainly due to the edges of the overlap region where the N and C terminal domains are located (blue and red markers on the stick-models in FIG. 7). In addition, type I collagen sequence analysis shows that most amino-acids in the sequence have one-sixth of the D-band repeat, i.e., a repeat of approximately 39 residues including phenylalanines. This means that in the model in FIG. 7, phenylalanine residues at the surface of neighbouring microfibrils have a higher probability to be in close proximity compared to phenylalanine residues in the canonical D-banded fibril. Phenylalanines and tyrosines are the only 2 natural amino-acids that are photocrosslinkable to another phenylalanine or tyrosine, respectively, by UVC irradiation in the absence of a photo-initiator. In other words, the washed collagen multifilament bundles should be sensitive to UVC crosslinking based on their molecular architecture.

### Thermogravimetric Analysis (TGA)

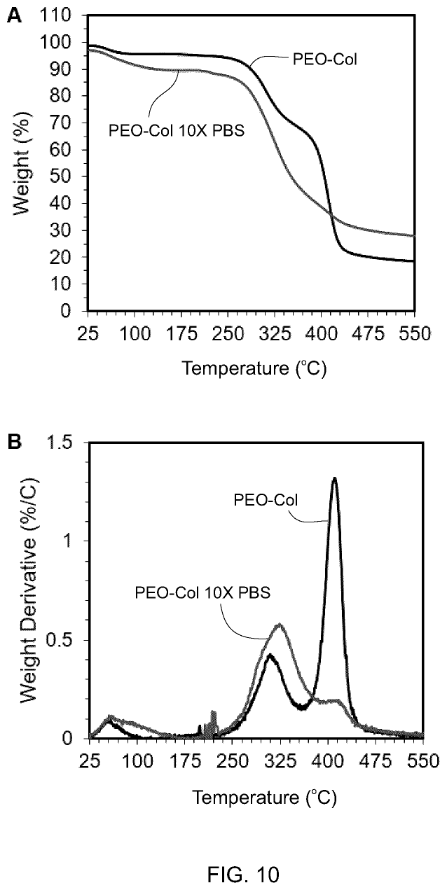

The thermal degradation of PEO-collagen multifilaments happens in a three-step degradation process, as seen by the presence of three peaks for differential thermogravimetry (DTG) (FIG. 10, panel B). In the thermogravimetry (TG) and differential thermogravimetry (DTG) curves, the first thermal degradation with a maximum degradation rate temperature of 58° C. can be assigned to the evaporation of unbound water (G. Ramanathan, et al. J. Biomater. Tissue Eng. 2014, 4, 203). The second peak in the DTG curves at a maximum degradation rate temperature of 310° C. is due to the decomposition of collagen (B. H. Leon-Mancilla, et al. J. Appl. Res. Technol. 2016, 14, 77). The third peak at a maximum degradation rate temperature of 411° C. corresponds to the degradation of PEO in the multifilament (N. S. Vrandečić, et al. Thermochim. Acta, 2010, 498, 71).

To estimate the percentage of PEO by weight in the washed multifilaments, the area of the PEO peak was estimated at 411° C. in the DTG curve (FIG. 10, panel B) and compared to the area of the 310° C. and 411° C. peaks in the DTG curve (FIG. 10, panel B). A PEO content of about 2.5% by weight was obtained using this method adapted from ASTM E1131-20, “Standard test method for compositional analysis by thermogravimetry”.

### Effect of Crosslinking on the Tensile Properties of Collagen Multifilament Bundles

To investigate the effect of crosslinking on ultimate tensile strength (UTS) and Young's modulus (E), collagen multifilament bundles hydrated with graded PEO: 10×PBS solutions were either irradiated with UVC or treated with 1.0% glutaraldehyde. UVC produces zero-length crosslinks in the absence of a photo-initiator, thus mimicking in vivo enzymatic crosslinks formed by the activity of lysyl-oxidase, whereas glutaraldehyde produces very good mechanical properties and resistance to biodegradation.

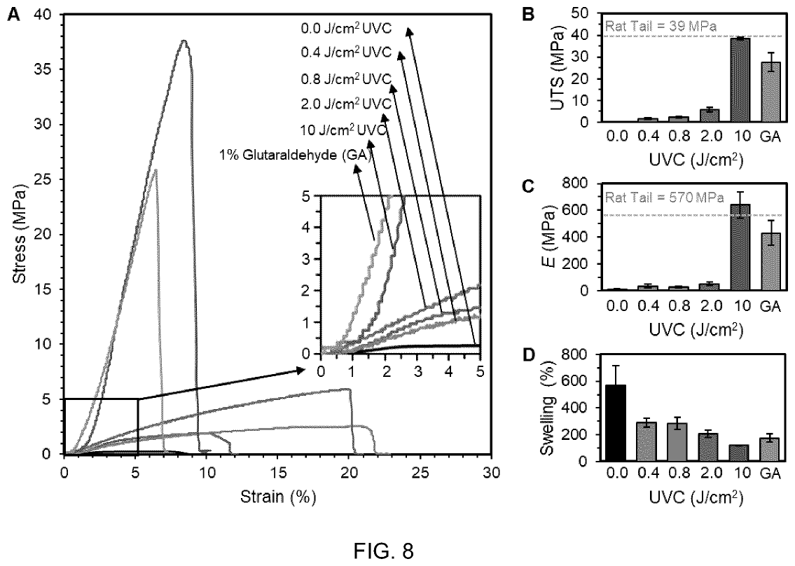

UVC treatment resulted a dramatic dose-dependant increase in the UTS and Young's modulus of the multifilament bundle (FIG. 8A, Panel to FIG. 8, Panel C), indicating that the number of crosslinks increases with UVC dose. At the highest UVC dose examined (10 J/cm²) the UTS and Young's modulus values exceeded those of the 1.0% glutaraldehyde treatment. Compared to collagen multifilament bundles that were not crosslinked, the 10 J/cm² UVC dose led to a 22-fold increase in UTS to reach 38.5±0.8 MPa (FIG. 8, Panel B), a value within the lower limit of a what has been reported for mammalian tendons. This UTS is similar to wet spun, glutaraldehyde crosslinked, collagen multifilaments (40 MPa) and also to the UTS reported for the rat tail tendon fibers (39 MPa). A similar trend was observed for the Young's modulus, which increased 19-fold relative to the collagen multifilament bundles that were not crosslinked to reach 638±95 MPa at a UVC dose of 10 J/cm² (FIG. 8, Panel C), and outperforming wet spun, glutaraldehyde crosslinked, collagen multifilaments (281 MPa). Swelling ratio was inversely proportional to UVC dose (FIG. 8, Panel D). Collagen multifilament bundles that were not crosslinked swelled by 572.7±141.8% within 1.5 hrs of incubation compared to only 120.1±4.3% for multifilament crosslinked with 10 J/cm² UVC (FIG. 8, Panel D). A statistical analysis comparing the UTS, Young's modulus, and swelling rations for various crosslinking conditions in presented in Table 1.

TABLE 1

| Crosslinking | | Young's | |
|---|---|---|---|
| Conditions | UTS (MPa) | Modulus (MPa) | Swelling (%) |
| 0 J/cm² UVC | 0.3 ± 0.0^{a} | 12.3 ± 1.5^{a} | 572.7 ± 141.8^{a} |
| 0.4 J/cm² UVC | 1.7 ± 0.4^{a} | 32.8 ± 14.1^{a} | 291.1 ± 35.7^{b} |
| 0.8 J/cm² UVC | 2.5 ± 0.4^{a} | 27.4 ± 4.9^{a} | 285.6 ± 43.2^{b} |
| 2.0 J/cm² UVC | 5.9 ± 1.1^{b} | 51.0 ± 12.9^{a} | 207.9 ± 28^{bc} |
| 10 J/cm² UVC | 38.5 ± 0.8^{d} | 638.6 ± 95.6^{c} | 120.1 ± 4.3^{c} |
| 1 wt % Glutaraldehyde | 27.6 ± 4.4^{c} | 430.3 ± 92.6^{b} | 175.5 ± 31^{bc} |

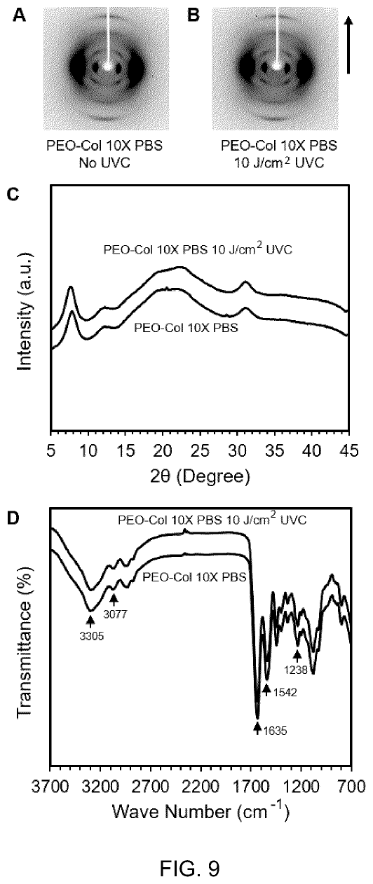

This is not the first attempt at crosslinking a collagen material using UV in the absence of a photo-initiator. Previous studies have shown that during UV treatment there is a competition between collagen crosslinking and collagen denaturation. In other words, the main limitation of UV in other collagen materials is that it denatures collagen at doses of 1 J/cm² or greater. FIG. 9 shows WAXS patterns and ATR-FTIR spectra for washed PEO-collagen multifilament bundles in graded PEO: 10×PBS solutions and washed PEO-collagen multifilament bundles in graded PEO: 10×PBS solutions after 10 J/cm² UVC to assess changes in the collagen structure. From the WAXS patterns in FIG. 9, Panel A to FIG. 9, Panel C, it is evident that there are no changes in peak shape and intensity between the non-crosslinked and the crosslinked PEO-collagen multifilament bundles, indicating that the spacing of the collagen triple helix and the distance between residues along the triple helix axis remains unchanged following UV crosslinking.

The ATR-FTIR spectra shown in FIG. 9, Panel D exhibit characteristic bands at 1635 cm⁻¹, 1542 cm⁻¹, and 1238 cm⁻¹ corresponding to the amide I, amide II and amide III bands, respectively, which are attributed to the presence of collagen in the multifilament. These bands are related to the C═O stretching mode, N—H bending vibrations of polypeptide chains, and coupled C—N stretching and N—H bending motions, respectively. An amide A band was also observed at 3305 cm⁻¹, which is due to N—H stretching, and finally, a weak absorption band for amide B was present at 3077 cm⁻¹, which is attributed to C—N stretching of peptide bonds. After UVC treatment there were no visible changes in the position and relative intensity of any of the bands (FIG. 9, Panel D), thus providing further evidence that the chemical composition of the collagen multifilament bundle was unchanged by the UVC crosslinking process.

Typically, the formation of crosslinks during UVC irradiation is attributed to the formation of free radicals on aromatic amino acid residues such as phenylalanine and tyrosine that absorb strongly in that range of wavelengths and represent less than 2% of amino acid residues in the collagen I molecule. These two residues likely react first with water molecules bound to the collagen molecules in the dry state to form phenoxyl and tyrosyl radicals that can react with neighbouring side chains of the same kind to form covalent bonds. Combining the lack of molecular-level structural and chemical change within multifilament exposed to 10 J/cm² UVC with the observed enhancement in tensile properties (FIG. 8) indicates that UVC treatment may create new C—C or C—N bonds within the multifilament. In addition to these qualitative findings, ATR-FTIR absorption ratio of amide III to 1450 cm⁻¹ band (1238 cm⁻¹/1450 cm⁻¹) were calculated for both crosslinked and non-crosslinked collagen multifilament bundles, which allows one to assess the degree of triple helix preservation. A ratio of about 1.0 corresponds to a properly folded triple helical conformation, whereas for the denatured collagen, gelatin, the ratio is about 0.6. Here, the ratios for crosslinked and non-crosslinked collagen multifilaments were 1.002 and 0.996, respectively. This is further evidence that UVC treatment did not disrupt the triple helices within the collagen. Collectively, these results support the structural model prediction that one-sixth of D-band staggering of microfibrils within the multifilament brings normally distantly-spaced phenylalanine residues into close enough proximity to allow for efficient UVC crosslinking without molecular damage.

Collagen monofilaments were prepared by multi-pin contact drawing of an entangled polymer solution consisting of acid-solubilized collagen and poly(ethylene oxide) (PEO). This approach produced thousands of PEO-collagen monofilaments at a time, each of up to 35 cm in length and between 1 and 5 μm in diameter, which were collected on a frame and consolidated into multifilament bundles that resemble tendon fascicles. The multifilament bundles were then hydrated in graded concentrations of PEO and PBS to promote assembly of collagen fibrils within each monofilament while preserving the structure of the multifilament bundle. Wide-angle X-ray scattering (WAXS) and attenuated total reflectance Fourier-transform infrared (ATR-FTIR) spectroscopy confirmed the presence of collagen and the formation of well-ordered collagen structures in the multifilament bundles after hydration. Hierarchical structural characteristics were further examined by small angle X-ray scattering (SAXS) and scanning and transmission electron microscopy. The SAXS and electron microscopy results were consistent with collagen fibrils containing microfibrils, staggered by exactly one-sixth of the microfibril D-band spacing to produce a periodicity of 11 nm. Sequence analysis predicted that in this structure, phenylalanine residues are close enough within and between microfibrils to become ultraviolet C (UVC) crosslinked. Phenylalanine crosslinking is not naturally occurring. In agreement with this analysis, the ultimate tensile strength and Young's modulus of the hydrated collagen multifilament bundles crosslinked by UVC radiation increased non-linearly with total UVC energy to reach values in the range of native tendons. The chemical and molecular structures of the hydrated collagen multifilaments was preserved after UVC crosslinking as demonstrated through ATR-FTIR and WAXS analysis further supporting the concept of phenylalanine specific crosslinking. This fabrication method recapitulates the structure of a tendon across multiple length scales and offers tunability in tensile properties using only collagen molecules and no other chemical additives in addition to PEO, which is removed during the hydration process.

The novel features will become apparent to those of skill in the art upon examination of the description. It should be understood, however, that the scope of the claims should not be limited by the embodiments but should be given the broadest interpretation consistent with the wording of the claims and the specification as a whole. Documents mentioned herein are hereby incorporated by reference.

1. A process for producing a multifilament collagen fiber bundle, the process comprising treating a polymer-collagen fiber bundle comprising a plurality of monofilaments of collagen supported on a support polymer with a series of liquid media having a decreasing concentration of the support polymer to remove the support polymer from the polymer-collagen fiber bundle, the support polymer being more soluble than the collagen in the liquid media.

2. The process of claim 1, further comprising producing the polymer-collagen fiber bundle by: contact drawing the support polymer and the collagen from a pre-strand composition containing the support polymer and the collagen to form polymer-collagen fibers; and bundling a plurality of the polymer-collagen fibers together to form the polymer-collagen fiber bundle.

3. The process of claim 1, wherein the treating comprises hydrating and the liquid media comprise aqueous media in which the support polymer is more soluble than the collagen.

4. The process of claim 3, wherein at least one aqueous medium of the series of aqueous media comprises a buffer.

5. The process of claim 1, wherein the series of aqueous media comprises a first aqueous medium comprising 10 wt % or less of the support polymer, based on total weight of the first aqueous medium.

6. The process of claim 5, wherein the first aqueous medium comprises 0.25-5 wt % of the support polymer.

7. The process of claim 1, wherein the series of liquid media comprises a first aqueous medium comprising 0.75-2 wt % of the support polymer, a second aqueous medium comprising 0.35-0.75 wt % of the support polymer and a third aqueous medium comprising 0.1-0.35 wt % of the support polymer, all weight percentages based on total weight of the respective aqueous media.

8. The process of claim 1, wherein the series of liquid media comprises a final aqueous medium in which the support polymer is absent.

9. The process of claim 5, wherein the polymer-collagen fiber bundle is treated for 1-24 hours in each aqueous medium of the series of liquid media.

10. The process of claim 1, wherein, at least in part, the decreasing concentration decreases continuously as the liquid medium is continuously replaced or diluted with other liquid medium having less and less concentration of the support polymer.

11. A multifilament collagen fiber bundle comprising collagen fibers having diameters of at least 0.3 μm, each collagen fiber comprising collagen fibrils of smaller diameter than the collagen fibers, each collagen fibril comprising collagen microfibrils of smaller diameter than the collagen fibrils and each collagen microfibril comprising a bundle of collagen molecules, the collagen microfibrils forming a hierarchical D-band structure in the collagen fibrils, wherein adjacent collagen microfibrils are shifted by ⅙^{th} of the D-band in comparison to microfibrils in naturally occurring collagen of a same type.

12. The multifilament collagen fiber bundle of claim 11, wherein the collagen molecules are type I, type III or type V collagen.

13. The multifilament collagen fiber bundle of claim 11, wherein the collagen molecules are atelomeric.

14. The multifilament collagen fiber bundle of claim 11, further comprising one or more additives.

15. The process of claim 1 further comprising irradiating the multifilament collagen fiber bundle produced therein with ultraviolet radiation at a total energy dose of 0.1 J/cm² or more to crosslink the multifilament collagen fiber bundle.

16. The process of claim 15, wherein the total energy dose is in a range of 5 J/cm² to 100 J/cm².

17. The process of claim 15 carried out in an absence of a photo-initiator.

18. The multifilament collagen fiber bundle of claim 11, which is crosslinked.

19. (canceled)

20. (canceled)

21. The multifilament collagen fiber bundle of claim 18 having an ultimate tensile strength of 1 MPa or greater and/or a Young's modulus of 20 MPa or greater.

22. A process for producing a multifilament collagen fiber bundle, the process comprising treating a polymer-collagen fiber bundle comprising a plurality of monofilaments of collagen supported on a support polymer with a series of liquid media having a decreasing osmotic pressure to remove the support polymer from the polymer-collagen fiber bundle, the support polymer being more soluble than the collagen in the liquid media.

23. (canceled)

24. (canceled)

25. (canceled)

26. (canceled)

27. (canceled)
